(19) United States
(12) Patent Application Publication
(10) Pub. No.: US 2026/0279386 A1
(43) Pub. Date: Sep. 17, 2026

(54) ANALYSIS OF AMBIENT SPEECH FOR HEALTH CONDITIONS USING VOCAL BIOMARKERS

(71) Applicant: Canary Speech, Inc., Provo, UT (US)

(72) Inventors: Nate Blaylock, Forney, TX (US); Henry O'Connell, Spanish Fork, UT (US); Jayden Stuver, Provo, UT (US); Jeff Adams, Tyngsborough, MA (US)

(73) Assignee: Canary Speech, Inc., Provo, UT (US)

(21) Appl. No.: 19/307,510

(22) Filed: Aug. 22, 2025

Related U.S. Application Data
(60) Provisional application No. 63/698,857, filed on Sep. 25, 2024.

(51) Int. Cl. G10L 25/66; A61B 5/00; G10L 17/06; G10L 25/27

(52) U.S. Cl. CPC G10L 25/66; A61B 5/4803; A61B 5/7267; A61B 5/7275; G10L 17/06; G10L 25/27

(57) ABSTRACT

Disclosed herein are techniques for diagnosing health conditions of a subject based on the subject's vocal biomarkers, including techniques for generating one or more models (e.g., machine learning models) trained to predict a health condition in multiple subjects. One or more techniques may include receiving ambient audio data, segmenting the ambient audio data by speaker to form speech data samples for a given speaker, filtering the speech data of the given speaker to remove irrelevant or insignificant portions, coalescing filtered speech data to form speech data that satisfies a predetermined threshold of speech data, analyzing the input speech data using one or more trained models, and outputting the prediction of whether the given speaker has any of the one or more health conditions.

### RELATED APPLICATIONS

This application claims the benefit under 35 U.S.C. § 119 (e) of U.S. Provisional Application Ser. No. 63/698,857, titled “AI-ENHANCED ANALYSIS OF AMBIENT SPEECH FOR HEALTH CONDITIONS USING VOCAL BIOMARKERS” and filed on Sep. 25, 2024, which is incorporated by reference herein in its entirety.

Some embodiments described herein may incorporate or leverage some of the subject matter of U.S. Pat. Nos. 10,152,988, 10,311,980, and/or U.S. Pat. No. 12,125,497, each of which is incorporated by reference herein in their entirety and at least for their discussions of identification and extraction of vocal biomarkers, training/configurating detectors of health conditions based on vocal biomarkers, and detection of health conditions based on vocal biomarkers.

### BACKGROUND

To receive an accurate diagnosis, subjects may undergo a comprehensive evaluation process that is often facilitated by a primary care physician or generalist, who assesses the subject's symptoms and determines the need for specialized care. If necessary, the subject is then referred to a specialist, such as a cardiologist, oncologist, or neurologist, who has advanced training and expertise in a specific area of medicine. The specialist may conduct further evaluation and testing to confirm or rule out a diagnosis.

### SUMMARY

In one embodiment, there is provided a method for predicting whether a subject has one or more health conditions. The method includes determining a prediction by analyzing ambient audio data that includes a plurality of speech samples of the subject having an aggregate duration greater than or equal to a threshold duration. The prediction is determined at least in part by analyzing the aggregated audio data using one or more trained models that were trained with audio data of a plurality of prior subjects along with corresponding information regarding whether each prior subject had one or more health conditions. The method further includes outputting the prediction of whether the subject has any of the health conditions.

In another embodiment, there is provided a computer-readable storage medium storing computer-executable instructions that, when executed by a processor, cause the processor to carry out a method. The method includes determining a prediction by analyzing ambient audio data that includes a plurality of speech samples of the subject having an aggregate duration greater than or equal to a threshold duration. The prediction is determined at least in part by analyzing the aggregated audio data using one or more trained models that were trained with audio data of a plurality of prior subjects along with corresponding information regarding whether each prior subject had one or more health conditions. The method further includes outputting the prediction of whether the subject has any of the health conditions.

In yet another embodiment, there is provided an apparatus comprising a processor and a storage medium having stored computer-executable instructions. When executed by the processor, the instructions cause the processor to perform a method. The method includes determining a prediction of whether a subject has one or more health conditions by analyzing ambient audio data that comprises a plurality of speech samples with an aggregate duration meeting or exceeding a threshold duration, wherein the analysis is based at least in part on one or more trained models previously trained with audio data of prior subjects and corresponding health condition information, and to output the prediction.

The foregoing is a non-limiting summary of the invention, which is defined by the attached claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a block diagram of a system with which one or more embodiments may operate;

FIG. 2 is an example audio processing facility, in accordance with one or more embodiments;

FIG. 3 is an example system that may be used to select features for training a machine learning model for diagnosing a health condition based on ambient audio data and then using the selected features to train the machine learning model;

FIG. 4 is a flowchart of a process that may be implemented in one or more embodiments to evaluate audio data for identifying features related to a health condition and generating a result that may be provided to a clinician to assist or inform the clinician's diagnosis;

FIG. 5 is a flowchart of a process that may be implemented in one or more embodiments to train one or more models to generate a result that may be provided to a clinician to assist or inform the clinician's diagnosis;

FIG. 6 is a block diagram of a computing device with which one or more embodiments may operate.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

Disclosed herein are techniques for evaluating the health of a subject (e.g., a patient), including techniques for generating one or more models (e.g., machine learning models) trained to predict (e.g., diagnose) one or more health conditions in a subject. Some such techniques include receiving audio data comprising speech of a subject and/or of one or more other persons, extracting vocal biomarkers of a subject from the ambient audio data, and determining whether the vocal biomarkers correspond (or are likely to correspond, or are sufficiently likely to correspond, or otherwise satisfy a criteria for correspondence) to potential presence of a health condition (or multiple health conditions) in the subject. Such extracting of vocal biomarkers and/or determining of presence of a health condition may be based at least in part on an analysis of the audio data of speech by one or more models, such as models generated or trained using machine learning. In some embodiments described herein, the audio data may be ambient audio data, which may be audio data that is captured from microphones disposed in the area of a speaker speaking to a listener (other than the microphone), such that the speaker is not speaking solely for capture by the microphone or for purposes of vocal biomarker assessment. In some such cases, for the example, the ambient audio data may include speech of multiple speakers participating in a conversation with one another and potentially others. The conversation may be in the context of a professional setting such as a professional meeting between the speakers, which may include a clinical encounter between a subject (e.g., patient) and a clinician (e.g., physician, nurse, technologist) who may be providing care to the subject. In such a setting, the speakers in the conversation may be speaking to one another and not to the microphone, and may be naturally speaking on one or multiple topics and may not be reading a prepared script or statement to be captured by the microphone. In some such embodiments, the ambient audio data may be continuously received during a course of the speaking (e.g., the conversation), and extracting vocal biomarkers from the ambient audio data may be performed contemporaneous with the speaking (e.g., in real or near-real time with capture of the speech of the subject). In some embodiments in which ambient audio data may include the speech of multiple individuals (e.g., a clinician and a subject, or other conversation participants), biomarkers may be extracted from the speech of multiple individuals participating in the conversation, and a determination of health conditions be made for each individual. In some embodiments, audio data may be collected over time, and receiving a threshold amount of acoustically informative audio data from an individual may trigger the extraction of vocal biomarkers from the ambient audio data associated with the individual (e.g., audio of that individual's speech, as opposed to audio data for other speakers). In other embodiments, vocal biomarkers may be extracted and analyzed over time during a course of speaking until a threshold amount of acoustically informative audio data has been captured and analyzed and/or until one or more other suitable conditions is met.

In operation, some techniques disclosed herein may continuously capture ambient audio data during clinical encounters and separate each speaker's speech through diarization. Filler utterances, utterances less than a threshold duration in length, audio that does not satisfy audio recording quality conditions (e.g., due to background noise higher than a threshold amount or volume or intensity, perceived distance from the microphone being higher than a threshold or volume of speech being below a threshold, or other conditions on audio quality), or other acoustically uninformative fragments may in some cases be filtered from the ambient speech data so that downstream processing focuses on speech data segments sufficiently acoustically informative to yield reliable vocal biomarkers. Once a pre-determined (e.g., a threshold) amount of acoustically informative speech data samples is accumulated for a given individual or other condition(s) are met, machine learning models, rules, and/or other techniques may be used extract vocal biomarkers (e.g., in real or near-real time) and the vocal biomarkers may be analyzed to determine probabilistic likelihoods of specific health conditions. It may be advantageous in some scenarios for the analysis to run unobtrusively (e.g., in the background) during a conversation with a subject and for care providers, clinicians, or others with a relationship with the subject to receive live, quantitative feedback regarding possible health conditions of the subject during the encounter. This may, in some cases, enable earlier intervention into the health conditions and/or more precise longitudinal tracking of potential health conditions due to ease of data capture and analysis over time. This may also shorten the time needed for visits from the clinician and/or patient perspective. In some embodiments in which data is captured of multiple speakers speaking (e.g., audio of a conversation), multiple (e.g., parallel) analyses may be run for each discernible speaker. This way, a workflow may flag potential health conditions for other speakers, such as potential care provider fatigue, while simultaneously surfacing subject-focused insights and may do so without interrupting the natural flow of the clinical encounter.

Health conditions for which some embodiments may operate include neurological disorders (e.g., Parkinson's disease, Alzheimer's disease, Lou Gehrig's disease, and stroke rehabilitation), mental health (e.g., depression, anxiety, post-traumatic stress disorder, and bipolar disorder), cardiovascular and respiratory conditions (e.g., chronic obstructive pulmonary disease (COPD), asthma, and cardiac stress), developmental disorders (e.g., autism spectrum disorder and speech delays), metabolic and endocrine disorders (e.g., obesity, diabetes, and thyroid dysfunction), behavioral state (e.g., aggression, emotion), pain level, wellness (e.g., stress, mood), risk assessment (e.g., risk of imminent violent behavior), impairment (e.g., by alcohol, drugs, sleepiness, mental or physical fatigue), and/or any other health condition (transient, temporary, chronic, or otherwise) that can be reflected in the speech of a patient.

In some embodiments, a determination of whether a health condition is present may be made when analysis indicates that the subject (the speaker) has the condition. Additionally or alternatively, in some embodiments a determination of whether a health condition is present may be made when analysis indicates that the subject (the speaker) is or may be developing the condition or has a health state in which the health condition could or is likely to develop. Some embodiments may operate in accordance with vocal biomarker analysis to identify whether a health condition is present by analyzing for signs of possible or potential health conditions of a speaker. A speaker may be determined to have a health condition when a confidence, likelihood, or probability analysis of one or more biomarkers in connection with a health condition meets a threshold, when multiple such thresholds are met by different biomarkers, and/or when other conditions are met, such that a determination of whether a subject has a health condition may be made when the subject is determined to have a potential health condition. In some embodiments, an output indicating that a subject may have a health condition may be made when the system determines that the health condition is more likely than not to be present. In other embodiments, however, such an output may be generated when the system determines that one or more conditions have been satisfied for a health condition, which may indicate that there is enough reason (e.g., out of an abundance of caution or otherwise) to prompt further investigation by the subject and a provider of a health condition, even if it has not necessarily been determined that the subject more likely than not has the health condition.

The inventors have recognized and appreciated that, conventionally, speech analysis has not been widely used for predicting health conditions and that a contributing factor to this has been the low resolution of the data available from traditional speech analytics. Higher data resolution often allows for more precise analysis and robust identification of subtle patterns associated with diseases or conditions, which leads to higher reliability. High resolution data for diagnostic purposes has not, however, been traditionally available from speech.

Traditional speech data has had low information density because they rely on analysis only of the words included in the speech and discard other aspects of the speech (e.g., the audio of the speaking of those words). The average rate of speech for the average English speaker is approximately 150 words per minute, which in such conventional systems yields approximately 150 data points per minute. This can result in what is often a mismatch between the available resolution of the data and analytical tools that are available for use in analyzing data so as to generate highly reliable results. For example, machine learning models, such as those based on some approaches to deep learning, are often designed to extract patterns from large and detailed datasets. When the input data is sparse or lacks the necessary resolution, these tools often underperform. For example, data of low resolution may result in overfitting or underfitting of a model, in which a model captures noise instead of meaningful patterns or fails to capture complexities altogether.

The inventors have recognized and appreciated that speech could be advantageously used in machine learning driven diagnostics of health conditions if data resolution could be increased. The inventors determined that, rather than a word-based analysis, if audio data of speech were instead subjected to an acoustic analysis of speech, this may increase data resolution. For example, where conventional word-based analysis methods may produce 150 data points per minute, acoustic analysis of speech may produce several million data points per minute. This increase in resolution can provide data points usable by some machine learning models to provide reliable diagnostic analysis of speech. Vocal biomarkers might be derivable from audio data of speech using such acoustic analysis, where the vocal biomarkers may include objective measures of voice such as pitch, pitch variability, tremors (including microtremors) in speech, tone, rhythm, amplitude, speech rate, prosody, pause duration, respiratory markers, and/or other features characterizing the acoustics of the speech. The inventors have additionally recognized and appreciated that such vocal biomarkers may be used to diagnose health conditions or assist clinicians in diagnosis of health conditions, such as onset or progression of health conditions. Such diagnosis may be done using vocal biomarkers as a symptom or indicator of such health conditions. In fact, the inventors have recognized and appreciated that vocal biomarkers may in some cases be used to detect early-stage health conditions such as in the case of some neurological conditions like Alzheimer's Disease, Parkinson's Disease, and others. Speech analysis may therefore offer an earlier detection or improved reliability of early detection of certain diseases, above what is conventionally available to providers and patients for some such diseases. Earlier detection could provide for better management of a health condition and improved patient outcomes. As a result of the difficulties noted above, though, speech has not traditionally been used, preventing the realization of these benefits for patients and providers.

The inventors have additionally recognized and appreciated that, even if speech data were to be used for these purposes, some techniques for analyzing a patient's speech data could introduce barriers to analyzing the speech data. For example, some conventional approaches require the patient to speak specific phrases and speak directly into specific devices such as microphones. This is often done so as to have a “control” sample for analysis, where the same words are spoken in the same way between patients, to minimize variability and in what was conventionally thought to be a way to increase reliability. The inventors have recognized and appreciated, however, that this approach might cause speakers to speak in a way different from their natural speech. A speaker's natural (e.g., spontaneous) speech may in some cases be more reliable for vocal biomarkers analysis, and it would be advantageous if techniques were available for capturing and analyzing natural speech of a subject.

The inventors additionally recognized that, beyond the reliability challenges, capturing specific speech (e.g., reading of a script) provided to a microphone or other device may also increase time for a clinical encounter. Clinicians already have precious little time to see patients and a large number of tasks to accomplish. Reserving time for a session in which a subject speaks to a microphone reading a script or prompt may detract from other work that could be done during an encounter between clinician and subject (e.g., patient encounter). Some clinicians may therefore skip a speech evaluation that requires dedicated time absent there being a specific factor driving the clinician to want the speech diagnostics. This can present a hurdle to early diagnosis of some health conditions when other symptoms are not yet present but which could be detected with speech. It would again be advantageous if a technique were available for capturing natural speech of a speaker without substantially impacting the existing flow of the encounter.

While the inventors have recognized and appreciated that acoustic analysis of speech and vocal biomarkers can be used to reliably detect health conditions in patients, the inventors have additionally recognized and appreciated that modern clinical encounters generate a wealth of audio data, such as in the dialogue between patient and care provider. Yet most of the acoustic nuance in these dialogues evaporate the moment it is spoken. Care providers may rely on memory, hurried note-taking, or post-visit transcription to recall speech characteristics that might signal early cognitive decline, depression, respiratory impairment, or even their own creeping fatigue. Ambient clinic noise, overlapping voices, and the time constraints further erode the chances that anyone will notice subtle vocal changes. Subjective human judgments can also introduce inconsistency and bias. As a result, treatment monitoring is imprecise and diagnoses are delayed.

While clinicians may trust their approaches to considering patient speech in a clinical encounter, the inventors recognize that most of the audio data generated in any given clinical encounter goes untapped and valuable clues, such as changes in pitch stability, articulation rate, respiratory pause patterns, or spectral tilt, are therefore left unmeasured, even though such vocal biomarkers can be as predictive as some laboratory tests. The inventors recognize that, with ambient speech data, vocal biomarker analysis might in some cases be performed based on and, in some cases, even contemporaneous with a conversation with a subject (e.g., a patient in an encounter with a clinician such as a physician). This may enable increased efficiency in the clinician's time by allowing the clinician to handle other aspects of the clinical encounter in parallel with vocal biomarker analysis.

In some embodiments and beyond clinical encounters, such tools may support continuous health monitoring. Ambient listening devices (e.g., home devices, smart speakers, smart phones, smart watches, etc.) may continuously monitor speech data, even in the context of a subject speaking by themselves (i.e., not in the context of a conversation with other speakers) or when the person is alone in the environment of a microphone but speaking with others (e.g., a phone call where the microphone can only detect one side of the phone conversation). This may provide longitudinal insights into a subject's health, capturing dynamic changes and enabling timely interventions. For example, subtle deterioration or other change in vocal biomarkers could trigger an alert for a clinical encounter (e.g., triggering the subject or a caregiver to schedule an appointment with a clinician, such as a physician) to evaluate the results of the speech analysis, capture additional speech for analysis, and/or otherwise analyze the subject. This may provide early intervention in progressive conditions like depression or cardiovascular disorders. This capability may ultimately lead to better outcomes and more efficient use of healthcare resources. Accordingly, while some examples described below are presented in the context of a clinical encounter, it should be appreciated that techniques described herein are not so limited. Techniques described herein may be used to evaluate speech of a subject for one or more health conditions outside of the context or setting of a clinical encounter.

Some techniques described herein may be useful in some embodiments in generating one or more models to output one or more health risk scores for one or more health conditions based on vocal biomarkers extracted from ambient speech data. Further described herein are examples of techniques and systems with which such techniques may be used. These include, for example, (1) systems with which some embodiments of the methods described herein may operate; (2) methods for conducting training of at least one model using information from one or more speakers (e.g., audio data of speech) corresponding to a presence of a health condition; (3) methods of identifying one or more health conditions predicted by the at least one model contemporaneous with speech (e.g., in real or near-real time); (4) methods of identifying when sufficient acoustically-informative audio of speech of a subject has been collected to output a determination of whether one or more health conditions are present in a subject.

The following description and examples illustrate in detail some embodiments of techniques and technologies described herein. It is to be understood that embodiments are not limited to acting in accordance with the specific examples provided herein, as other approaches are possible. Those of skill in the art will recognize that there may be variations and modifications from the specific examples below that are within the scope of this disclosure.

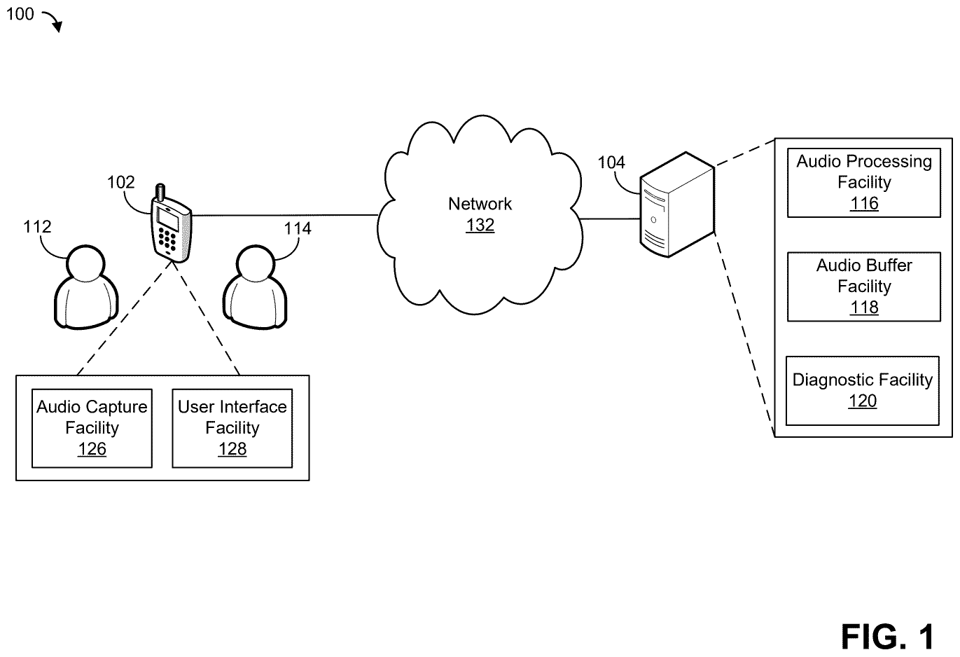

FIG. 1 is a block diagram of a system 100 with which one or more embodiments may operate. The system 100 may be used by a clinician 112 (e.g., a physician, nurse, researcher, technologist, technician, etc.) and/or a subject 114 (e.g., a patient, clinical study participant, etc.) to diagnose the subject 114 with, or as part of a diagnostic evaluation of the subject 114 by a clinician for, one or more health conditions based on the subject's vocal biomarkers from, for example, a clinical encounter with the subject 114. The subject 114 may be a human subject that can provide speech. Health conditions with which some embodiments described herein may operate may include, for example, neurological disorders (e.g., Parkinson's Disease, Alzheimer's Disease, mild cognitive impairment (MCI), amyotrophic lateral sclerosis (ALS), and multiple sclerosis (MS)), mental health and behavioral disorders (e.g., depression, anxiety disorders, bipolar disorder, schizophrenia and psychotic disorders), cardiovascular and respiratory diseases (e.g., chronic obstructive pulmonary disease (COPD), heart failure, hypertension, sleep apnea), developmental disorders (e.g., autism spectrum disorder, speech and language delays, Huntington's Disease), metabolic and endocrine disorders (e.g., diabetic neuropathy, thyroid disorders, obesity and metabolic syndrome), infections and autoimmune diseases (e.g., respiratory infections, Lupus, rheumatoid arthritis), and/or any other speech-manifesting health conditions. The system 100, in some embodiments, may be used to produce a degree of likelihood of the subject 114 having the one or more health conditions by analyzing audio data of speech with one or more trained machine learning (ML) models. The ML model(s) may have been trained on prior audio data from other subjects with one or more diagnosed health conditions.

The audio data that the system 100 can analyze may be ambient audio data that includes speech or may be data that characterizes or relates to audio data of speech, such as that was derived through an acoustic analysis of speech.

The system 100 can include a client computing device 102, which may be a desktop or laptop computer, smart mobile phone, tablet, wearable (e.g., smart watch, device on a lanyard, smart glasses, or other wearable), server, or suitable device. The client computing device 102 may include an audio capture facility 126 and/or a user interface facility 128.

The audio capture facility 126 may connect with or operate one or more sensors for capturing audio, such as a microphone or microphone array, by which the facility 126 may receive audio data of speech. Such a microphone/array may be integrated with the client computing device 102 or separate from it and communicatively connected via wired and/or wireless communication. A microphone/array may, in some cases, be disposed in a room in which a conversation is taking place, such as an exam room or other room of a medical office in a case of a patient encounter. A microphone may be mounted on or integrated with a wall, ceiling, furniture, or other surface, or be worn by a clinician or subject. Embodiments are not limited to operating with a particular type of microphone.

In some embodiments, the audio capture facility 126 may trigger recording ambient audio when a non-speech signal is provided (e.g., a button to begin ambient audio capture), when specific speech is detected (e.g., a trigger word or wake-up word), when any speech is detected, or upon another suitable condition. In some embodiments, the audio capture facility 126 is configured to preprocess captured ambient audio data. For instance, the audio capture facility 126 may preprocess the ambient audio data to filter out noise (e.g., by analyzing a signal-to-noise ratio of the audio data and removing outlier or noisy data), remove speech segments that are less than a threshold duration (e.g., to remove one- or two-word utterances), utterances that are not acoustically informative or otherwise meet one or more conditions, and/or the like.

The captured audio may be stored on the client computing device 102. Speech may be captured in any suitable manner. For example, the audio capture facility 126 may recorded speech provided by subject 114 or by a clinician 112. In some cases, the speech may be speech spoken by the subject 114 in response to a prompt. In other cases, the speech may be speech spoken by the subject 114 during a discussion with clinician 112, such as a dialogue between the clinician 112 and subject 114. Such dialogue may be a health-related conversation, such as a patient discussing with a care-providing clinician a reason the patient is pursuing care, and/or may be another conversation between a clinician and a subject such as discussing the weather, sports, family life, or other topics. In some such cases, the captured ambient audio data may be audio of two or more speakers, and in some cases filtering or speaker segmentation/diarization may be used to yield audio data for speech of the subject 114.

The user interface facility 128 enables the subject 114 or the clinician 112 to interact with the client computing device 102. The subject 114 or the clinician 112 can use the user interface facility 128 to provide data to the client computing device 102 such as credentials (which may be used to access data results of a diagnostic session), initiate a diagnostic session with the diagnostic device 104, and/or any other input. The subject 114 or the clinician 112 can also use the user interface facility 128 to receive and/or display data by the client computing device 102 such as diagnostic results (which may be received from the diagnostic device 104) and/or any other output. The user interface facility 128 may be in any suitable format. In some examples it may be a web interface, such as one or more web pages into which values may be output and which may display results of a diagnostic analysis by the diagnostic device 104, but embodiments are not so limited. Other embodiments may use a mobile application, software application, or other software, firmware, or other computer instructions. The user interface facility 128 may accept input in a variety of different formats, such as through speech recognition, text input, or other means, as embodiments are not limited in this respect.

The system 100 may include a diagnostic device 104, which may be a desktop or laptop personal computer, mobile device (e.g., smart mobile phone, tablet), server, or other suitable device or set of devices. The diagnostic device 104 may include an audio processing facility 116, an audio buffer facility 118, and/or a diagnostic facility 120.

The audio processing facility 116 is configured to process ambient audio (e.g., pre-captured or captured in real time, such as from an audio capture facility 126), identify speech data in the ambient audio, identify different speakers, and/or process speech data from the ambient audio to determine one or more vocal biomarkers. When ambient audio is received, the audio processing facility 116 may perform speech detecting, discriminating between speech and non-speech data of the ambient audio data to filter out background noise, silence, or other irrelevant acoustic data. The audio processing facility 116 may also perform speaker diarization, segmenting the speech data of the ambient audio data by individual speaker. The audio processing facility 116 may also filter the speech data of one or more speakers to remove segments that are unlikely to contribute meaningful information for biomarker analysis. This may include brief utterances, filler words, or speech artifacts that lack sufficient prosodic, acoustic, or temporal complexity. This may additionally or alternatively include speech that does not include multiple different words, or where a meaning of words used in the speech does not satisfy at least one criterion (e.g., having a non-trivial meaning, such as expressing more than mere agreement (“yes” or “yes yes yes”) or mere disagreement (“no” or “no no no”)). Additionally or alternatively, audio that may not contribute meaningful information for biomarker analysis may include audio for which acoustic quality does not pass one or more criteria, such as having a signal-to-noise threshold below a threshold, being too quiet, or other criterion related to the acoustics of the audio. Generally speaking, a determination of whether segments of audio data are to be used for determining whether a subject has one or more health conditions may include evaluating whether segments of audio do (or do not) satisfy one or more conditions. The audio processing facility 116 may also subject the speech data of one or more speakers to feature extraction, where the speech data is transformed into speech data embeddings, which are multidimensional representations that encode certain vocal attributes. The embeddings may include representations of prosodic features (e.g., pitch, intonation, and rhythm), acoustic features (e.g., formant frequencies, spectral energy, and harmonics), temporal features (e.g., speech rate and pause duration), respiratory features (e.g., breath control or voice tremor), and/or other features indicative of vocal biomarkers.

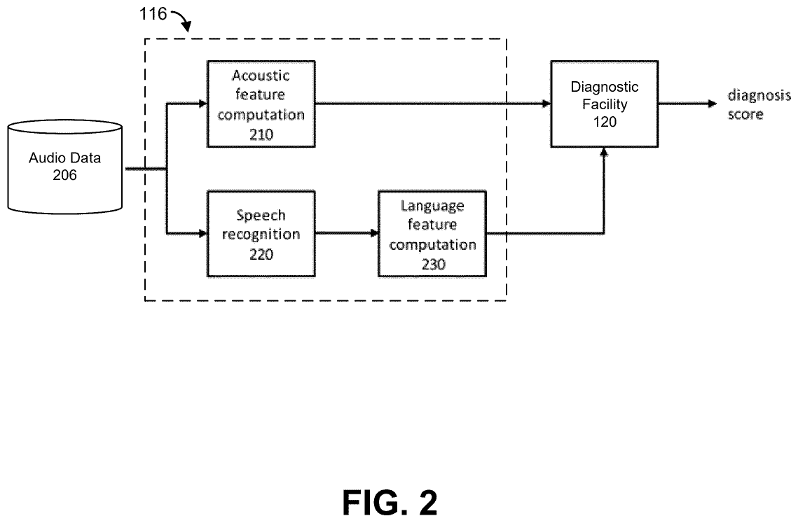

As discussed above, vocal biomarkers include measurable indicators from a subject 114 of some biological state and/or condition of the user. The biological state of the user may include the presence of a health condition and the condition of the user may include the user's quality of life. Vocal biomarkers may include objectively identifiable characteristics such as prosodic features, acoustic features, temporal features, and/or respiratory features in the speech data. Vocal biomarkers may be used to identify potential health conditions of the subject 114. Illustrative techniques for identifying vocal biomarkers are discussed in further detail below with respect to FIG. 2.

In some embodiments, the audio processing facility 116 may perform role recognition to determine a relationship between different speakers in ambient audio data. For example, the audio processing facility 116, based on the context of the conversation, the sentiment or tone of the conversation, and/or the like, may determine whether one speaker is in a dominant role to the other speaker (e.g., in an employer/employee relationship, in a doctor/patient relationship, in a parent/child relationship) or whether the speakers are colleagues, friends, strangers, and/or the like.

In some embodiments, the audio processing facility 116 may use a machine learning model to determine the different speakers in the conversation and the role relationship between the speakers. For instance, the audio processing facility 116 may train a machine learning model to identify different speakers. The audio processing facility 116, for instance, may provide at least a portion of the captured sound to a machine learning model that has been trained to perform speaker recognition, role recognition, and/or the like.

The audio processing facility 116 may work in coordination with the audio buffer facility 118, which is configured to receive speech data from the audio processing facility 116. The audio buffer facility 118 may manage the accumulation and organization of speech data over time, enabling downstream processes (e.g., those involving biomarker analysis) to operate with a sufficient amount of acoustically informative speech data input. When ambient audio data is continuously obtained (e.g., captured by the audio capture facility 126), the audio buffer facility 118 may work with the audio processing facility 116, receiving speech data, which may have been segmented by speaker and/or filtered to remove irrelevant or insignificant content, such as short utterances or filler words. Rather than treating the incoming audio as a transient stream, the audio buffer facility 118 may retain and coalesce the filtered speech segments into a structured repository that grows incrementally as new data is received. This helps address the challenge of data sparsity in clinical environments, where an individual may speak only intermittently or in brief exchanges that, on their own, are insufficient for vocal biomarker analysis.

In some embodiments, the audio buffer facility 118 may maintain an organized (e.g., indexed) structure of the speech data, tagged by speaker identity, timestamps, and/or associated metadata. When the audio processing facility 116 is ready to perform vocal biomarker extraction, this organization enables the audio processing facility 116 to have access to a consolidated body of significant speech data that meets the pre-determined threshold (e.g., 30 seconds) for accurate and reliable analysis. In this way, the audio buffer facility 118 may be a passive data store and an active enabler of diagnostic quality by ensuring that the audio processing facility 116 and/or diagnostic facility 120 are operating on sufficient speech data.

The diagnostic facility 120 is configured to generate a prediction of whether the subject 114 has one or more health conditions. To do so, the diagnostic facility 120 may receive the outputs (e.g., embeddings) of the audio processing facility 116 and perform an analysis for predicting a diagnosis of the subject 114. Generating diagnoses based on the outputs of the audio processing facility 116 is described in further detail below with respect to FIG. 2.

The diagnostic facility 120 may evaluate whether the vocal biomarkers (e.g., extracted from speech data) correspond to known vocal biomarkers (e.g., acoustic or prosodic features) of specific health conditions. These conditions may include, but are not limited to, anxiety, depression, Alzheimer's disease, Parkinson's disease, cardiovascular stress, fatigue, and other neurocognitive or emotional conditions. The diagnostic facility 120 may utilize classification and/or regression models that have been trained on extensive labeled datasets, allowing it to assess both the presence and severity of these conditions with a high degree of confidence.

The diagnostic facility 120 may provide the outputs to a clinician 112, to a subject 114, or to another interested party (e.g., a caregiver or an insurance company). The diagnostic facility 120, for instance, may provide the results in an interface (e.g., a graphical user interface). For example, the diagnostic facility 120, during a conversation between a clinician 112 (e.g., a physician) and subject 114 (e.g., a patient), may present the analysis results on the clinician's device while the conversation is ongoing (e.g., contemporaneous with the conversation, such as during the conversation or while at least one of the speakers is still located where the conversation was held or within 30 seconds or 1 minute or 5 minutes of the end of the conversation, or in real-time). The results may indicate that a health condition for the patient has been detected, may provide a likelihood that the patient has the health condition, or the like, based on the patient's speech during the conversation.

In some embodiments, the results output from the diagnostic facility 120 may include one or more scores. The scores may reflect a confidence level or a degree of accuracy of the outcomes of the audio data analysis. In some embodiments, the scores may include a score associated with an analysis of one duration of audio data and another score associated another analysis of different duration of audio data. The scores may be combined into a single score, such as an average, a weighted average, a sum, or the like. The diagnostic facility 120 may provide the score(s) to a doctor, a patient, or the like.

In some embodiments, the ambient audio data and/or the results may be provided as part of the electronic health record (EHR) of the subject 114. For instance, the diagnostic facility 120 may upload the ambient audio data between the clinician 112 and the subject 114, may create and upload a transcription of the ambient audio data, may upload the analysis results including the likelihood that the subject 114 has various medical conditions, may upload information about the machine learning models that were used to perform the analysis and/or the like, to become part of the electronic health record of the subject 114. Such an upload may be performed using, for example, one or more application programming interfaces (APIs) for an EHR, or other interface for interacting with an EHR.

In some embodiments, the diagnostic facility 120 is located on, integrated with, or otherwise part of a chatbot or AI assistant. In such an embodiment, the diagnostic facility 120 may run in the background to analyze a speaker's speech, voice, sentiment, and/or the like during the conversation between the speaker and the chatbot. The diagnostic facility 120, based on the analysis of the speaker's speech, may notify the speaker of the likelihood that the speaker has a medical condition, may notify the speaker's doctor, may notify a caregiver for the speaker, may notify emergency services, and/or the like.

The system 100 can include a network 132 to facilitate communications among the client computing device 102 and/or the diagnostic device 104. The network 132 can be or include any one or more wired and/or wireless, local- and/or wide-area networks (which may be physical and/or virtual), including one or more enterprise networks and/or the Internet. The network 132 includes one or more servers, routers, switches, and/or other networking equipment.

While the example of FIG. 1 includes the client computing device 102 and the diagnostic device 104 as separate devices, embodiments of the disclosure are not so limited and may include greater or fewer than the number of devices shown. In some embodiments, the system 100 may include one or more devices for each of the client computing device 102 and the diagnostic device 104. For example, the diagnostic device 104 may be a cluster of devices on a cloud platform. In some embodiments, the operations performed by multiple facilities may be performed by a single facility, and vice versa. For example, the diagnostic facility 120 may perform the operations of the audio processing facility 116. In some embodiments, the operations performed by multiple devices may be performed by a single device, and vice versa.

While for ease of description the example of FIG. 1 was discussed in the context of a clinical encounter, and speaker was described as a clinician, it should be appreciated that embodiments are not limited to operating with audio data of a clinical encounter or otherwise between a clinician and a subject/patient. In other embodiments, the two speakers may have any of a variety of other roles, including no role such as in the case of a social conversation. Other potential conversations with which some embodiments described herein may operate include conversations between caller and call center agent, conversation between teacher and student, conversations between customer and store employee, or other conversations.

For example, in some embodiments, the audio capture facility 126 and the diagnostic facility 120 are located on, integrated with, or otherwise part of a call center system. In such an embodiment, the audio capture facility 126 may “listen” to persons on a telephone call, e.g., the person(s) at the call center and the person(s) on the other end of the call and analyze the speech of at least one person during the conversation and the diagnostic facility 120 may then analyze a speaker's speech, voice, sentiment, and/or the like during the conversation between the speaker and the call center system (e.g., a human call center agent, an interactive voice response (IVR) system, an automated agent, or other party). The diagnostic facility 120, based on the analysis of the speaker's speech, may notify the speaker of the likelihood that the speaker has a medical condition, may notify a call center agent of the same, or may notify the speaker's clinician or another clinician or a caregiver, may notify emergency services, and/or the like.

As another example, the audio capture facility 126 may be disposed in or use a microphone or device with a microphone, such as a smart speaker a speaker's residence, disposed at a subject's residence, place of care (e.g., hospital inpatient, nursing home, rehabilitation facility, physical therapy center, or other care center), place of work, or other physical location. Or, the facility 126 may be disposed it or use a device worn or carried by the speaker (e.g., a smart phone, tablet, wearable, or other device). Audio may be captured by the device and transferred to a diagnostic facility 120, which may be integrated with a service provider with which the speaker has a subscription or in another service accessible by the device of the speaker. The diagnostic facility 120 may analyze the speaker's speech, voice, sentiment, and/or the like during speech (e.g., the speaker speaking or vocalizing alone, or as a part of a conversation between the speaker and another person, who in some cases may also be in the presence of the microphone and whose speech may also be captured). The diagnostic facility 120, based on the analysis of the speaker's speech, may notify the speaker of the likelihood that the speaker has a medical condition, may notify a call center agent of the same, or may notify the speaker's clinician or another clinician or a caregiver, may notify emergency services, and/or the like. In some cases, such a system may be useful for longitudinal tracking of a user's health, such as by capturing and analyzing speech of a subject over hours, days, weeks, months, or years, and iteratively sampling and analyzing speech to determine whether the subject has one or more health conditions. Techniques described below may be used to conduct such iterative speech analysis.

FIG. 2 is a block diagram depicting of an example system for processing speech data with a model to predict a diagnosis, in accordance with one or more embodiments. In processing the speech data, features may be computed from the speech data, and then the features may be processed by the model. Any appropriate type of features may be used.

The features may include acoustic features, where acoustic features are any features computed from the audio data that do not involve or depend on performing speech recognition on the audio data (e.g., the acoustic features do not use information about the words spoken in the speech data). For example, acoustic features may include mel-frequency cepstral coefficients, perceptual linear prediction features, jitter, or shimmer.

The features may include language features where language features are computed using the results of a speech recognition. For example, language features may include a speaking rate (e.g., the number of vowels or syllables per second), a number of pause fillers (e.g., “ums” and “ahs”), the difficulty of words (e.g., less common words), or the parts of speech of words following pause fillers.

The audio data 206 (e.g., from the audio capture facility 126 or the audio buffer facility 118) may be processed by acoustic feature computation facility 210 and/or speech recognition facility 220. Acoustic feature computation facility 210 may compute acoustic features from the audio data 206, such as any of the acoustic features described herein. Speech recognition facility 220 may perform automatic speech recognition on the audio data using any appropriate techniques (e.g., Gaussian mixture models, acoustic modelling, language modelling, and neural networks). In some embodiments, the speech recognition facility 220 may use pretrained embedding models based on the audio signal.

Because speech recognition facility 220 may use acoustic features in performing speech recognition, some processing of these two components may overlap and thus other configurations are possible. For example, acoustic feature computation facility 210 may compute the acoustic features needed by speech recognition facility 220, and speech recognition facility 220 may thus not need to compute any acoustic features. In some embodiments, acoustic feature computation facility 210 may use various techniques for voice activity detection to detect that a person is speaking.

Language feature computation facility 230 may receive speech recognition results from speech recognition facility 220 and process the speech recognition results to determine language features, such as any of the language features described herein. The speech recognition results may be in any appropriate format and include any appropriate information. For example, the speech recognition results may include a word lattice that includes multiple possible sequences of words, information about pause fillers, and the timings of words, syllables, vowels, pause fillers, or any other unit of speech.

The features computed by acoustic feature computation facility 210 and/or language feature computation facility 230 may be the vocal biomarkers identified by the audio processing facility 116.

The performance of diagnostic facility 120 may depend on the features computed by acoustic feature computation facility 210 and/or language feature computation facility 230. Further, a set of features that performs well for one health condition may not perform well for another health condition. For example, word difficulty may be a feature for diagnosing Alzheimer's disease but may not be useful for determining if a person has a concussion. For another example, features relating to the pronunciation of vowels, syllables, or words may be useful for Parkinson's disease but may be less useful for other health conditions. Accordingly, techniques are needed for determining a first set of features that performs well for a first health condition, and this process may need to be repeated for determining a second set of features that performs well for a second health condition.

The selection of features for diagnosing a health condition may be more important in situations where an amount of training data for training the machine learning model is relatively small. For example, for training a machine learning model for diagnosing concussions, the needed training data may include audio data of a number of individuals shortly after they experience a concussion. Such data may exist in small quantities and obtaining further examples of such data may take a significant period of time.

Training machine learning models with a smaller amount of training data may result in overfitting where the machine learning model is adapted to the specific training data but because of the small amount of training data, the model may not perform well on new data. For example, the model may be able to detect all of the concussions in the training data but may have a high error rate when processing production data of people who may have concussions.

One technique for preventing overfitting when training a machine learning model is to reduce the number of features used to train the machine learning model. The amount of training data needed to train a model without overfitting increases as the number of features increases. Accordingly, using a smaller number of features allows models to be built with a smaller amount of training data.

Where it is beneficial to train a model with a smaller number of features, it may be advantageous to select the features that will allow the model to perform well. For example, when a large amount of training data is available, hundreds of features may be used to train the model and it is more likely that appropriate features have been used. Conversely, where a small amount of training data is available, only 10 or so features may be used to train a model, and it is more important to select the features that are most important for diagnosing the health condition.

Described below are some examples of features that may be used to diagnose a health condition, in some embodiments. It should be appreciated that embodiments are not limited to operating with all of these features or with any particular combination of these features. Other embodiments may use other features.

Acoustic features may be computed using short-time segment features. When processing audio data, the duration of the audio data may vary. For example, some audio may be a second or two and other audio may be several minutes or more. For consistency in processing audio data, it may be processed in short-time segments (sometimes referred to as frames). For example, each short-time segment may be 25 milliseconds, and segments may advance in increments of 10 milliseconds so that there is a 15 millisecond overlap over two successive segments.

Short-time segment features may in some cases include one or more of the following examples: spectral features (such as mel-frequency cepstral coefficients or perceptual linear predictives); prosodic features (e.g., pitch, energy, or probability of voicing); voice quality features (e.g., jitter, jitter of jitter, shimmer, or harmonics-to-noise ratio); entropy (e.g., to capture how precisely an utterance is pronounced where entropy may be computed from the posteriors of an acoustic model that is trained on natural speech data).

The short-time segment features may be combined to compute acoustic features for the audio. For example, a two-second speech sample may produce 200 short-time segment features for pitch that may be combined to compute one or more acoustic features for pitch.

In some cases, short-time segment features may be combined to compute an acoustic feature for a speech sample. For example, in some implementations, an acoustic feature may be computed using statistics of the short-time segment features (e.g., arithmetic mean, standard deviation, skewness, kurtosis, first quartile, second quartile, third quartile, the second quartile minus the first quartile, the third quartile minus the first quartile, the third quartile minus the second quartile, 0.01 percentile, 0.99 percentile, the 0.99 percentile minus the 0.01 percentile, the percentage of short-time segments whose values are above a threshold (e.g., where the threshold is 75% of the range plus the minimum), the percentage of segments whose values are above a threshold (e.g., where the threshold is 90% of the range plus the minimum), the slope of a linear approximation of the values, the offset of a linear approximation of the values, the linear error computed as the difference of the linear approximation and the actual values, or the quadratic error computed as the difference of the linear approximation and the actual values). In some implementations, an acoustic feature may be computed as a speech embedding to represent the partial or full audio. The speech embedding may include identity vectors such as an i-vector or an x-vector of the short-time segment features and speech representation based on a self-supervised pre-trained model, such as wav2vec or Trillson. An identity vector may be computed using any appropriate techniques, such as performing a matrix-to-vector conversion using a factor analysis technique and a Gaussian mixture model for an i-vector or a neural network model for an x-vector.

Language features may in some cases include one or more of the following examples of features. A speaking rate, such as by computing the duration of all spoken words divided by the number of vowels or any other appropriate measure of speaking rate. A number of pause fillers that may indicate hesitation in speech, such as (1) a number of pause fillers divided by the duration of spoken words or (2) a number of pause fillers divided by the number of spoken words. A measure of word difficulty or the use of less common words. For example, word difficulty may be computed using statistics of 1-gram probabilities of the spoken words, such as by classifying words according to their frequency percentiles (e.g., 5%, 10%, 15%, 20%, 30%, or 40%). The parts of speech of words following pause fillers, such as (1) the counts of each part-of-speech class divided by the number of spoken words or (2) the counts of each part-of-speech class divided by the sum of all part-of-speech counts.

In some embodiments, language features may include a determination of whether a person answered a question correctly. For example, a person may be asked what the current year is or who the President of the United States is. The person's speech may be processed to determine what the person said in response to the question and to determine if the person answered the question correctly. Further, in some embodiments, language features may include a determination whether a person read correctly, e.g., read a presented passage correctly. In such an embodiment, the word error rate is computer and compared to the expected reading script, e.g., using an automatic speech recognition (ASR) result. In some embodiments, when the question prompt is intended to assess a verbal fluency test, e.g., asking the user to list the words in a category such as animals, an evaluation is performed to determine if the user's response actually belongs to the expected category by checking or calculating the distance between word vectors.

To train a model for diagnosing a health condition, a corpus of training data (a “training corpus” or “training data”) may be collected. The training corpus may include examples of audio data (e.g., ambient audio data) where the diagnosis of the subject 114 is known. For example, the rows of a table of may correspond to database entries. In this example, each entry includes an identifier of a person, the known diagnosis of the person (e.g., no concussion or a mild, medium, or severe concussion), and a filename of a file that contains the audio data. The training data may be stored in any appropriate format using any appropriate storage technology.

The training corpus may store a representation of audio data of a subject using any appropriate format. For example, an audio data item of the training corpus may include digital samples of an audio signal received at a microphone (e.g., of an audio capture facility 126) or may include a processed version of the audio signal, such as mel-frequency cepstral coefficients.

A single training corpus may contain audio data relating to multiple health conditions, or a separate training corpus may be used for each health condition (e.g., a first training corpus for concussions and a second training corpus for Alzheimer's disease). A separate training corpus may be used for storing audio data for people with no known or diagnosed health condition, as this training corpus may be used for training models for multiple health conditions.

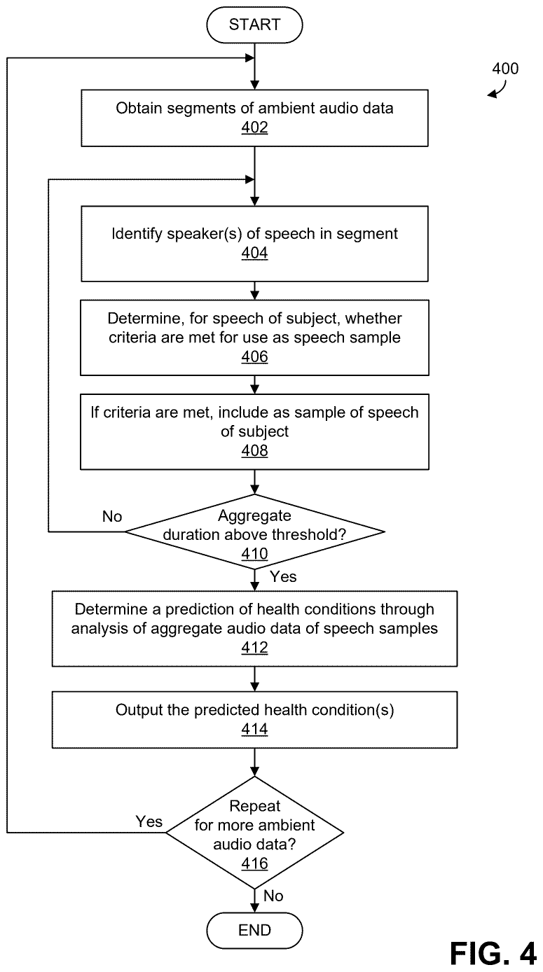

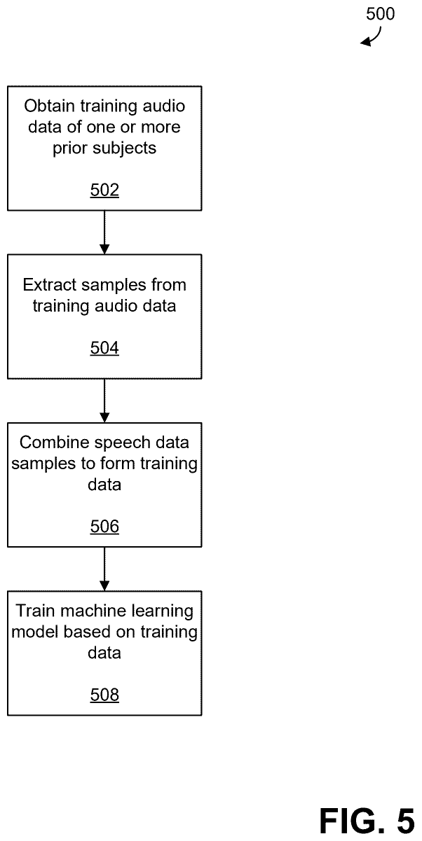

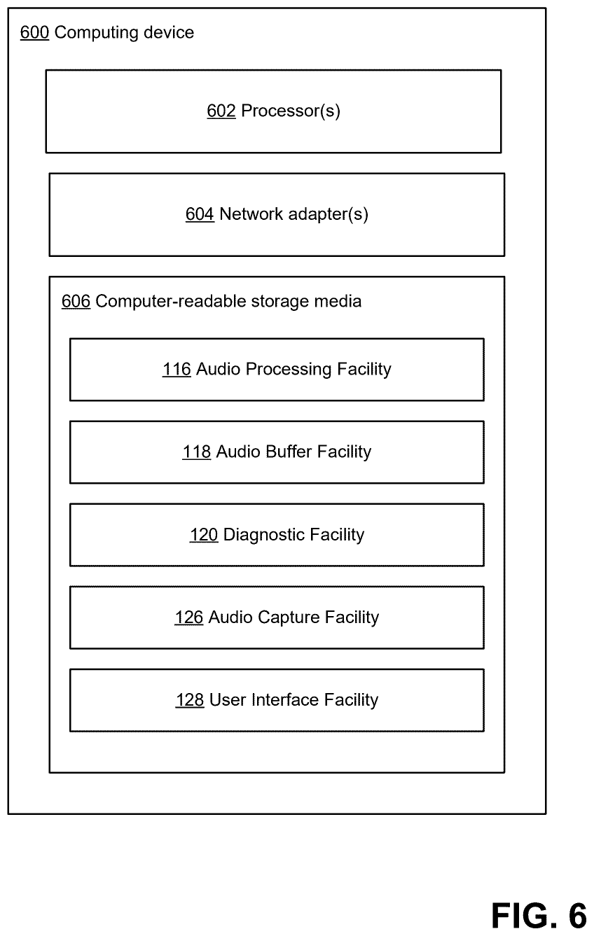

The diagnostic facility 120 may process the features (including acoustic features and/or language features) with one or more machine learning models to output one or more diagnosis scores that indicate whether the subject 114 has a health condition described herein, such as a score indicating a probability that the subject 114 has the health condition and/or a score indicating a severity of the health condition. The health condition may, in some cases, be a specific type of a health condition in the case of a health condition with multiple types. The diagnostic facility 120 may use any appropriate techniques, such as a classifier implemented with a support vector machine or a neural network, such as a multi-layer perceptron, a fully-connected dense network, a convolutional neural network, and/or the like. Generating a diagnostic prediction is described in further detail below with respect to FIGS. 4 and 6. Some examples of training a machine learning model to generate a diagnostic prediction is described in further detail below with respect to FIG. 5.

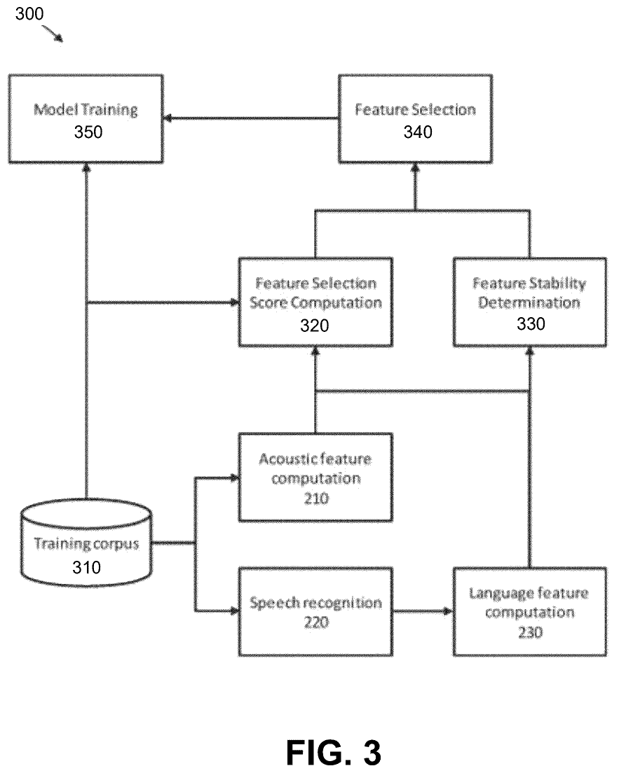

FIG. 3 depicts an example system 300 that may be used to select features for training a machine learning model of the diagnostic facility 120 for diagnosing a health condition based on audio data and using the selected features to train the machine learning model of the diagnostic facility 120. In some embodiments, system 300 may be used in different instances or different iterations to select features for different health conditions. For example, a first use of system 300 may select features for diagnosing concussions and a second use of system 300 may select features for diagnosing Alzheimer's disease (or other health conditions).

System 300 includes a training corpus 310 of audio data items for training a machine learning model for diagnosing a health condition. Training corpus 310 may include any appropriate information, such as audio data of multiple people with and without the health condition, a label indicating whether or not person has the health condition, and any other information described herein.

Acoustic feature computation facility 210, speech recognition facility 220, and/or language feature computation facility 230 may be implemented as described above to compute acoustic and language features for the audio data in the training corpus. Acoustic feature computation facility 210 and language feature computation facility 230 may compute a large number of features so that the best performing features may be determined. This may be in contrast to the example of FIG. 2 where these components are used in a production system and thus these components may compute only the features that were previously selected.

Feature selection score computation component 320 may compute a selection score for each feature (which may be an acoustic feature, a language feature, or any other feature described herein). To compute a selection score for a feature, a pair of numbers may be created for each audio data item in the training corpus, where the first number of the pair is the value of the feature, and the second number of the pair is an indicator of the health condition diagnosis. The value for the indicator of the health condition diagnosis may have two values (e.g., 0 if the person does not have the health condition and 1 if the person has the health condition) or may have a larger number of values (e.g., a real number between 0 and 1 or multiple integers indicating a likelihood or severity of the health condition). Accordingly, for each feature, a pair of numbers may be obtained for each audio data item of the training corpus.

Feature selection score computation component 320 may compute a selection score for a feature using the pairs of feature values and diagnosis values. Feature selection score computation component 320 may compute any appropriate score that indicates a pattern or correlation between the feature values and the diagnosis values. For example, feature selection score computation component 320 may compute a Rand index, an adjusted Rand index, mutual information, adjusted mutual information, a Pearson correlation, an absolute Pearson correlation, a Spearman correlation, or an absolute Spearman correlation.

The selection score may indicate the usefulness of the feature in detecting a health condition. For example, a high selection score may indicate that a feature should be used in training the machine learning model, and a low selection score may indicate that the feature should not be used in training the machine learning model.

Feature stability determination component 330 may determine if a feature (which may be an acoustic feature, a language feature, or any other feature described herein) is stable or unstable. To make a stability determination, the audio data items may be divided into multiple groups, which may be referred to as folds. For example, the audio data items may be divided into five folds. In some implementations, the audio data items may be divided into folds such that each fold has an approximately equal number of audio data items for different genders and age groups.

The statistics of each fold may be compared to statistics of the other folds. For example, for a first fold, the median (or mean or any other statistic relating to the center or middle of a distribution) feature value (denoted as Mₗ) may be determined. Statistics may also be computed for the combination of the other folds. For example, for the combination of the other folds, the median of the feature values (denoted as Mₒ) and a statistic measuring of variability of the feature values (denoted as Vₒ), such as interquartile range, variance, or standard deviation, may be computed. The feature may be determined to be unstable if the median of the first fold differs too greatly from the median of the second fold. For example, the feature may be determined to be unstable if:

$${M}_{1} < {M}_{o} - C \frac{{V}_{o}}{2} or {M}_{1} > {M}_{o} + C \frac{{V}_{o}}{2}$$

where C is a scaling factor. The process may then be repeated for each of the other folds. For example, the median of a second fold may be compared with median and variability of the other folds as described above.

In some implementations, if, after comparing each fold to the other folds, the median of each fold is within a predetermined threshold from the median of the other folds, then the feature may be determined to be stable. Conversely, if the median of any fold is outside the predetermined threshold from the median of the other folds, then the feature may be determined to be unstable.

In some implementations, feature stability determination component 330 may output a Boolean value for each feature to indicate whether the feature is stable or not. In some implementations, stability determination component 330 may output a stability score for each feature. For example, a stability score may be computed as the largest distance between the median of a fold and the other folds (e.g., a Mahalanobis distance).

Feature selection component 340 may receive the selection scores from feature selection score computation component 320 and the stability determinations from feature stability determination component 330 and select a subset of features to be used to train the machine learning model. Feature selection component 340 may select several features having the highest selection scores that are also sufficiently stable.

In some implementations, the number of features to be selected (or a maximum number of features to be selected) may be set ahead of time. For example, a number N may be determined based on the amount of training data, and N features may be selected. The selected features may be determined by removing unstable features (e.g., features determined to be unstable or features with a stability score below a threshold) and then selecting the N features with the highest selection scores.

In some implementations, the number of features to be selected may be based on the selection scores and stability determinations. For example, the selected features may be determined by removing unstable features, and then selecting all features with a selection score above a threshold.

In some implementations, the selection scores and stability scores may be combined when selecting features. For example, for each feature a combined score may be computed (such as by adding or multiplying the selection score and the stability score for the feature) and features may be selected using the combined score.

Model training component 350 may then train a machine learning model using the selected features. For example, model training component 350 may iterate over the audio data items of the training corpus, obtain the selected features for the audio data items, and then train the machine learning model using the selected features. In some implementations, dimension reduction techniques, such as principal components analysis or linear discriminant analysis, may be applied to the selected features as part of the model training. Any appropriate machine learning model may be trained, such as any of the machine learning models described herein.

In some implementations, other techniques, such as wrapper methods, may be used for feature selection or may be used in combination with the feature selection techniques presented above. Wrapper methods may select a set of features, train a machine learning model using the selected set of features, and then evaluate the performance of the set of features using the trained model. Where the number of possible features is relatively small and/or training time is relatively short, all possible sets of features may be evaluated, and the best performing set may be selected. Where the number of possible features is relatively large and/or the training time is a significant factor, optimization techniques may be used to iteratively find a set of features that performs well. In some implementations, a set of features may be selected using system 300, and then a subset of these features may be selected using wrapper methods as the final set of features.

FIG. 4 is a flowchart of a process 400 that may be implemented in one or more embodiments to evaluate audio data for identifying features related to a health condition and generating a diagnostic result. For explanatory purposes, the figure is described with reference to the system 100 of FIG. 1 and thus the process 400 may be a computer-implemented method. However, this is merely illustrative, and features of the system 100 may be performed by any other system for implementing the subject technology. The operations of the process 400 need not be performed in the order shown, and one or more operations of the process 400 need not be performed or can be replaced by other operations.

For ease of description, the division of processing between different computing devices is not addressed in connection with FIG. 4. But those skilled in the art will understand that the functionality may be divided between devices. Such a division between devices may, in some embodiments, include a division between edge devices or devices local to a speaker or to a clinician or caregiver, and devices that are remote such as in a data center. For example, voice activity detection (VAD), capture of ambient audio data, and at least some processing (e.g., speech detection and noise filtering) may be performed on an edge/local devices and analysis for prediction of one or more health conditions may be performed on one or more remote devices. Embodiments are not limited to a specific division of functionality between devices.

At operation 402, the audio buffer facility 118 obtains ambient audio data. The audio buffer facility 118 may continuously or intermittently receive ambient audio data over time. Obtaining ambient audio data may involve obtaining audio recordings from various contexts where speech data is generated, such as clinical encounters between doctors and patients, phone calls between call center agents and callers, or other conversational settings. The audio data may be sourced from pre-recorded audio files (e.g., uploaded via the user interface facility 128) or captured during interactions (e.g., by the audio capture facility 126), such as for contemporaneous (including real-time) analysis of speech data. Such contemporaneous capture and analysis of speech may be performed during the conversation or, in the case of a patient encounter, during the patient encounter, such that a result of a diagnostic analysis (or results of iterative diagnostic analyses) may be presented to one or more speakers during the conversation. Or, in the case of a single speaker rather than a conversation (e.g., home monitoring or personal monitoring), to the speaker close in time to capture of the speech. In some cases, a contemporaneous analysis of speech may be performed such that outputs are presented within 30 seconds, within 1 minute, within 5 minutes, or within another suitable window of the capture of the last speech that was analyzed to produce a prediction of whether a speaker has one or more health conditions.

In the context of clinical encounters, the audio data may be collected during consultations between care providers and patients. For example, a patient discussing symptoms with a physician or answering diagnostic questions may result in ambient audio data including speech that reflects vocal biomarkers associated with specific health conditions. An audio capture facility 126 may capture the ambient audio data using microphones integrated into clinical equipment, wearable devices, or ambient listening systems installed in the consultation room.

Similarly, in the context of phone calls between call center agents and callers, an audio capture facility may obtain ambient audio data from recorded customer service interactions. For instance, a caller calling a support line to report an issue or seek assistance may exhibit vocal biomarkers indicative of health conditions. The diagnostic device 104 may access the recordings through call center systems that store audio files for quality assurance or training purposes. The audio capture facility 126 may also or instead capture real-time audio during ongoing calls.

The diagnostic device 104 may also obtain ambient audio data from other conversational settings, such as interviews or group discussions, where the subject was speaking to another person within range of a microphone. Additionally, ambient monitoring systems, such as smart speakers or wearable devices, may record speech data throughout the day (e.g., continuously or at a sampling rate), capturing natural interactions that reflect the subject's vocal characteristics in various contexts.

In some embodiments, the audio processing facility 116 may preprocess the ambient audio data to enhance the quality of the ambient audio data. Preprocessing may include normalization to adjust the amplitude of the audio signal, noise cancellation to remove background interference, and/or vocal amplification to enhance the audibility of the speaker's voice. For example, in a clinical encounter, the audio capture facility 126 may filter out certain ambient sounds such as the hum of medical equipment or conversations from nearby rooms. Similarly, in a phone call scenario, the audio capture facility 126 may suppress static or line noise to focus on the speaker's voice. Additionally, non-speech portions of the audio, such as silence or irrelevant sounds (e.g., coughing or chair creaking), may be removed so that the samples are primarily speech. Once the ambient audio data is obtained, the audio processing facility 116 may obtain (e.g., extract) one or more segments of the ambient audio data that include a speech sample. The audio processing facility 116 may divide the ambient audio data into discrete segments of ambient audio data including a speech sample, where each speech sample corresponds to one or more utterances. The audio processing facility 116 may utilize techniques such as voice activity detection (VAD) to identify the start and end points of each utterance so that the speech samples are accurately segmented.

In some cases, the determination of whether a segment of ambient audio data includes speech may be performed before other preprocessing. In some cases, a great deal of ambient audio data may be obtained may include segments that do not contain audio of speech. In some such cases, more audio data or much more audio data may be obtained that does not include speech data than includes speech data. Accordingly, obtained segments may in some cases be analyzed by an audio processing facility to determine whether the segments include audio of speech. If so, other preprocessing may be done in some cases (e.g., noise cancellation) and/or the speech of the segments may be further analyzed.

At operation 404, the audio processing facility 116 analyzes each segment of ambient audio data to identify one or more speakers in each segment. The audio processing facility 116 may identify one or more speakers in each segment through diarization.

An approach to diarization may involve role recognition, which assigns roles to speakers based on the context and/or content of the conversation. For example, in a clinical encounter, the audio processing facility 116 may transcribe the audio data into text and use NLP to analyze the text for linguistic patterns and contextual cues. The audio processing facility 116 can then assign roles such as “doctor” and “patient” based on the distinct ways these types of individuals typically communicate. For instance, a doctor's speech may include medical terminology and diagnostic questions, while a patient's speech may consist of symptom descriptions and personal health concerns.

Another approach to diarization may involve utilizing input channels. This approach may be used in phone call scenarios, where the audio data is captured separately for each participant. Other cases may involve multiple microphones, where each microphone captures a different speaker and ambient audio data includes a channel for each speaker. Some such cases may leverage the different channels for speaker identification. For instance, in a call center setting, the audio processing facility 116 may attribute audio for a channel for a caller's speech/audio input to the subject/caller and audio for a channel for the agent's speech/audio input to the call center agent. By using the distinct audio streams from each input channel and identifying a speaker or role corresponding to each channel, the audio processing facility 116 can accurately segment the ambient audio data without requiring additional processing to distinguish between speakers.

Another approach to diarization may involve voice prints or vocal signatures. Voice prints may be or include unique acoustic characteristics associated with an individual's speech, such as pitch, tone, and cadence. The audio processing facility 116 may analyze acoustic characteristics to identify and differentiate speakers in the ambient audio data. For example, if a clinical encounter involves a doctor and a patient, the device may use pre-recorded voice samples and/or real-time voice analysis to match a speaker's voice print to their respective role.

Once the speech data samples are assigned to a particular speaker, the diagnostic device 104 can focus the remainder of the analysis on the segments of ambient audio data associated with the relevant speaker. For example, in a clinical encounter, the diagnostic device 104 may prioritize the patient's speech data for vocal biomarker analysis while disregarding the doctor's speech. Similarly, in a call center interaction, the diagnostic device 104 may analyze the caller's speech data to assess while disregarding the agent's speech.

At operation 406, the audio processing facility 116 may evaluate each segment to determine whether at least some audio data of the speech of the subject included within the segment satisfies one or more criteria for use as a speech sample in vocal biomarker analysis.

The audio processing facility 116 may evaluate segments to determine whether the segments are sufficiently long. In some embodiments, the audio processing facility 116 may apply criteria such as minimum duration thresholds (e.g., three seconds) so that the samples are long enough to capture meaningful vocal patterns.

The audio processing facility 116 may evaluate segments to determine whether they include sufficiently meaningful speech samples. In some embodiments, segments of ambient audio data with one- or two-word utterances may be discarded because those segments lack sufficient acoustic or prosodic complexity for meaningful analysis. For example, a brief response like “yes” or “no” may not provide enough information about pitch, tone, or rhythm to extract reliable biomarkers. In some embodiments, segments of ambient audio data that include utterances that lack meaning (e.g., gibberish or non-sensical phrases) may be discarded.

The audio processing facility 116 may evaluate segments to determine whether their speech samples have excessive noise. In some embodiments, excessive noise includes overlapping speech and/or background noise. For instance, in a group discussion setting, if multiple participants speak simultaneously, the audio processing facility 116 may discard the segments of ambient audio with overlapping speech samples to focus on those with clear, isolated utterances.

The audio processing facility 116 may evaluate segments to determine whether they have adequate audio quality. In some embodiments, inadequate audio quality includes segments of audio data that include distorted or muffled speech or high noise. For example, if a patient's voice is obscured by a malfunctioning microphone during a clinical encounter, the audio processing facility 116 may discard that segment to maintain the integrity of the analysis.

It should be understood that segment length, the meaning of speech samples, noise, and audio quality are merely examples and that other quality criteria are contemplated.

At operation 408, after evaluating the segments of speech for the relevant criteria, the audio processing facility 116 combines the samples (if more than one segment) that satisfy the criteria as part of the aggregated audio data of speech of the subject.

In some embodiments, the audio processing facility 116 may add a buffer between samples before combining them. The buffer may help ease any abrupt changes in tone, volume, and/or cadence that could otherwise disrupt the continuity of the input speech data. For example, if one sample ends with a loud, emphatic statement and the next sample begins with a soft, hesitant response, the buffer can help normalize the transition to create a more cohesive audio segment. The buffer may include a brief pause (e.g., 50 ms) or a gradual adjustment in volume levels.

This operation may involve continuously reviewing incoming or available segments and, for each segment that meets the established standards (e.g., sufficient duration, sufficient audio quality, and absence of overlapping speech or excessive background noise) adding the sample to the set of one or more samples intended for vocal biomarker analysis.

For example, if a segment of ambient audio data includes the subject speaking clearly for five seconds without interruption or significant background noise, and the utterance is more than a simple affirmation or negation, the audio processing facility 116 may include this segment in the set. Similarly, if another segment includes a longer response with diverse word usage and low noise, it may also be selected to be added to the set and/or aggregated with other segments.

At operation 410, the audio processing facility 116 may continue to add speech data samples to the aggregated speech data (e.g., operations 404-408) until the aggregated speech data satisfies a predetermined threshold duration so that the input speech data is sufficiently informative for vocal biomarker analysis.

The threshold length may be determined based on the requirements of the machine learning model and/or the nature of the analysis. For instance, the audio processing facility 116 may combine samples to form input data points of approximately 30 to 40 seconds in duration, as this length may be sufficient to capture meaningful vocal biomarkers such as pitch variability, prosody, and pause duration. If the aggregated segments are shorter than the threshold length, the audio processing facility 116 may continue to add additional segments until the threshold length is satisfied.

At operation 412, the diagnostic device 104 determines a prediction of whether a subject has any of one or more health conditions by analyzing speech data of the subject.

Once the aggregated speech data is prepared, the diagnostic device 104 may provide the aggregated speech data to one or more trained models to extract vocal biomarkers from the speech data, such as in the manner described above with respect to FIG. 2. These vocal biomarkers may include measurable features such as pitch, pitch variability, tone, rhythm, amplitude, speech rate, pause duration, and/or spectral characteristics. The extraction process may involve feature computation techniques, such as short-time segment analysis, statistical aggregation, and embedding generation using pre-trained models.

The diagnostic device 104 may provide the extracted vocal biomarkers to the diagnostic facility 120 where they may be analyzed using one or more trained machine learning models. These models may be developed using training data that includes prior audio data from prior subjects (e.g., patients), along with labels indicating whether each subject had one or more health conditions, which is described in further detail below with respect to FIG. 5. The training process may involve selecting features that are most predictive of specific health conditions so that the models are optimized for accuracy and reliability, as described above with respect to FIG. 3. The one or more machine learning models evaluate the vocal biomarkers to identify patterns or correlations indicative of health conditions, such as neurological disorders, mental health issues, cardiovascular stress, or respiratory impairments.

At operation 414, the diagnostic device 104 outputs the prediction of whether the subject has any of the one or more health conditions. Outputting the prediction may include presenting the results in a format accessible to relevant stakeholders, such as clinicians, subjects, and/or other authorized parties. The output may include a detailed analysis of the subject's speech data, highlighting the likelihood and/or severity of specific health conditions based on the extracted vocal biomarkers. The diagnostic device 104 may provide the predictions to the client computing device 102 to be displayed via a user interface facility 128, such as a clinician's desktop, tablet, or smartphone, enabling real-time access to diagnostic insights during a clinical encounter. In some embodiments, the diagnostic device 104 may also provide an indication of an activity in which the subject was engaged when the analyzed samples were captured.

The prediction may include one or more scores that quantify the confidence level and/or severity of the detected health conditions. For example, the diagnostic device 104 may provide a probability score indicating the likelihood that the subject has a particular condition and/or a severity score reflecting the extent of the condition. These scores may be derived from the analysis of the vocal biomarkers and may be presented alongside visual aids, such as charts, graphs, or tables, to facilitate interpretation. For example, a chart may display a probability score in a time series.

In some embodiments, the diagnostic device 104 integrates the prediction into the subject's EHR. This integration may include uploading the analyzed speech data, a transcription of the audio, the diagnostic results, and/or metadata about the machine learning models used in the analysis.

In some embodiments, the diagnostic device 104 may provide the prediction in real-time during ongoing interactions, such as a conversation between a clinician and a subject. This allows clinicians to receive live feedback on potential health conditions without interrupting the natural flow of the encounter. In some embodiments, the prediction may be delivered through automated systems, such as chatbots or call center platforms, where the results can be used to inform next steps, such as recommending further clinical evaluation.

At operation 416, the operations 402-414 may be performed iteratively over time. After each iteration, where a prediction is generated based on the most recent set of aggregated samples of speech, the audio buffer facility 118 may continue to obtain new ambient audio data. As new audio data becomes available, the audio processing facility 116 may analyze its segments, identify relevant speakers, evaluate the quality and/or content of the speech, and aggregate additional samples as appropriate and the diagnostic facility 120 may determine and output a prediction of health conditions based on the aggregated samples.

This iterative approach may be implemented using a sliding window of time. That is, the prediction may be determined using a first set of segments of ambient audio data in one iteration of the process, and the prediction may be determined using a second set of segments of ambient audio data in the next iteration of the process, where a set includes one or more segments or portions thereof. In some embodiments, the first and second set of segments may overlap. In some embodiments, the second set of segments may be a completely different set of segments obtained after the first set of segments.

For example, as each new segment is processed, the audio processing facility 116 may update the aggregated set of speech samples by including the most recent qualifying segments and, if desired, removing the oldest segments to maintain a consistent window duration. In this way, the analysis remains current and responsive to changes in the subject's speech patterns over time.

The process 400 may repeat in this iterative manner until the monitoring session is terminated, providing ongoing, up-to-date insights into the subject's health status. In some embodiments, the process 400 may repeat in this iterative manner according to a predetermined schedule (e.g., once every four hours). Running the process 400 according to a predetermined schedule may also control when microphones are capturing ambient audio data.

FIG. 5 is a flowchart of a process 500 that may be implemented in one or more embodiments to train one or more models to generate a diagnostic result. For explanatory purposes, the figure is described with reference to the system 100 of FIG. 1 and thus the process 500 may be a computer-implemented method. However, this is merely illustrative, and features of the system 100 may be performed by any other system for implementing the subject technology. The operations of the process 500 need not be performed in the order shown, and one or more operations of the process 500 need not be performed or can be replaced by other operations. For purposes of this description, the diagnostic device 104 trains the one or more models to generate a diagnostic result; however, it is contemplated that other devices may train the one or more models and the trained one or more models may be provided to the diagnostic device 104 for inference. At operation 502, the diagnostic device 104 obtains prior ambient audio data of one or more prior subjects. Obtaining prior audio data may involve obtaining audio recordings from various contexts where speech data is generated, such as clinical encounters between doctors and patients, phone calls between call center agents and callers, or other conversational settings. The prior audio data may be sourced from pre-recorded audio files or captured in real-time during interactions.

In the context of clinical encounters, the prior audio data may be collected during consultations between care providers and patients. For example, a patient discussing symptoms with a physician or answering diagnostic questions may generate speech data that reflects vocal biomarkers associated with specific health conditions. An audio capture facility 126 may capture the conversational audio using microphones integrated into clinical equipment, wearable devices, or ambient listening systems installed in the consultation room.

Similarly, in the context of phone calls between call center agents and callers, an audio capture facility 126 may obtain audio data from recorded customer service interactions. For instance, a caller calling a support line to report an issue or seek assistance may exhibit vocal characteristics indicative of health conditions. The diagnostic device 104 may access the recordings through call center systems that store audio files for quality assurance or training purposes. The audio capture facility 126 may also or instead capture real-time audio during ongoing calls.

The diagnostic device 104 may also obtain prior audio data from other conversational settings, such as interviews, group discussions, and/or ambient monitoring systems. For example, audio captured during a focus group discussion may provide insights into the vocal biomarkers of participants with known health conditions. Ambient monitoring systems, such as smart speakers or wearable devices, may continuously record audio data throughout the day, capturing natural interactions that reflect the subject's vocal characteristics in various contexts.

At operation 504, the audio processing facility 116 extracts speech data samples from the prior audio data. Extracting samples may include dividing the prior audio data into discrete speech data samples, where each speech data sample corresponds to one or more utterances. An utterance may be a continuous segment of speech spoken by a single speaker without interruption. For example, in a clinical encounter, an utterance might be a patient describing their symptoms in a single sentence, while in a call center interaction, an utterance could be a customer asking a question or providing feedback. The audio processing facility 116 may utilize techniques such as VAD to identify the start and end points of each utterance so that the samples are accurately segmented.

In some embodiments, after extracting the speech data samples from the prior audio data, the audio processing facility 116 may filter the speech data samples to remove those that are not conducive to (e.g., reduce the quality of) vocal biomarker analysis. Samples that are too short, such as one- or two-word utterances, may be discarded because they lack sufficient acoustic or prosodic complexity for meaningful analysis. For example, a brief response like “yes” or “no” may not provide enough information about pitch, tone, or rhythm to extract reliable biomarkers.

Similarly, samples with excessive noise or overlapping speech may be excluded to prevent interference with the analysis. For instance, in a group discussion setting, if multiple participants speak simultaneously, the audio processing facility 116 may discard the overlapping segments and focus on clear, isolated utterances. Samples with poor audio quality, such as those with distorted or muffled speech or high signal to noise ratio, may be removed from the dataset. For example, if a patient's voice is obscured by a malfunctioning microphone during a clinical encounter, the audio processing facility 116 may exclude that segment to maintain the integrity of the analysis. Additionally, the audio processing facility 116 may apply criteria such as minimum duration thresholds (e.g., three seconds) so that the samples are long enough to capture meaningful vocal patterns.

In some embodiments, the audio processing facility 116 segments prior speech data samples (or prior ambient audio data) by speaker so that the analysis focuses on the relevant subject's speech. This segregation may be achieved through diarization.

An approach to diarization may involve role recognition, which assigns roles to speakers based on the context and/or content of the conversation. For example, in a clinical encounter, the audio processing facility 116 may transcribe the prior audio data into text and use NLP to analyze the text for linguistic patterns and contextual cues. The audio processing facility 116 can then assign roles such as “doctor” and “patient” based on the distinct ways these types of individuals typically communicate. For instance, a doctor's speech may include medical terminology and diagnostic questions, while a patient's speech may consist of symptom descriptions and personal health concerns.

Another approach to diarization may involve utilizing input channels to segment speech data samples. This approach may be used in phone call scenarios, where the audio data is captured separately for each participant. For instance, the audio processing facility 116 may attribute the caller's input to the caller and the agent's input to the call center agent. By using the distinct audio streams from each input channel, the audio processing facility 116 can accurately separate the speech data samples without requiring additional processing to distinguish between speakers.

Another approach to diarization may involve voice prints or vocal signatures to assign roles to speakers. Voice prints may be or include unique acoustic characteristics associated with an individual's voice, such as pitch, tone, and cadence. The audio processing facility 116 may analyze acoustic characteristics to identify and differentiate speakers in the audio data. For example, if a clinical encounter involves a doctor and a patient, the device may use pre-recorded voice samples and/or real-time voice analysis to match a speaker's voice print to their respective role.

Once the speech data samples are assigned to a particular speaker, the diagnostic device 104 can focus the remainder of the analysis on the relevant speaker's samples. For example, in a clinical encounter, the diagnostic device 104 may prioritize the patient's speech data for vocal biomarker analysis while disregarding the doctor's speech. Similarly, in a call center interaction, the diagnostic device 104 may analyze the caller's speech data to assess emotional states such as stress or frustration.

At operation 506, the audio processing facility 116 combines speech data samples of the prior audio data of prior subjects to form training data points for training data. Combining samples may involve aggregating individual speech data samples into larger, cohesive units that each satisfy a threshold length. The threshold length may be determined based on the requirements of the machine learning model and/or the nature of the analysis. For instance, the audio processing facility 116 may combine samples to form training data points of approximately 30 to 40 seconds in duration, as this length may be sufficient to capture meaningful vocal biomarkers such as pitch variability, prosody, and pause duration. If the samples are shorter than the threshold length, the audio processing facility 116 may continue to add additional samples until the threshold length is satisfied. For example, if a prior patient's speech data includes three utterances of 10 seconds each, the audio processing facility 116 may combine these utterances to form a single training data point of 30 seconds.

In some embodiments, the audio processing facility 116 utilizes a sliding window approach to combine samples. The sliding window approach may involve creating overlapping training data points of speech data samples, where each training data point meets the threshold length but includes portions of the previous training data point. For example, if the threshold length is 30 seconds and there are 60 seconds of combined speech data samples, the audio processing facility 116 may create a first training data point from 0 to 30 seconds, a second training data point from 10 to 40 seconds, and so on.

At operation 508, the diagnostic device 104 trains a machine learning model based on the training data. The diagnostic device 104 labels each training data point with a label corresponding to the known condition of the subject. For example, in a clinical encounter, the training data points may be labeled with health conditions such as depression, anxiety, Parkinson's disease, or cardiovascular stress. These labels may serve as ground truth for the machine learning model, allowing it to learn the relationship between the vocal biomarkers present in the audio data and the corresponding condition.

The diagnostic device 104 may train separate machine learning models for different contexts to account for the distinct characteristics of each scenario. For instance, a model trained on audio data from doctor-patient interactions may focus on identifying health conditions such as neurological disorders or respiratory impairments, while a model trained on agent-customer interactions may prioritize emotional states and behavioral patterns.

During training, the diagnostic device 104 may use the labeled training data to optimize the parameters of the machine learning model. Optimization may involve feeding the training data into the model and adjusting its parameters (e.g., weights and biases) to optimize an objective function, such as minimizing the error between the model's predictions and the ground truth labels. For example, if the model predicts that a subject has depression based on their vocal biomarkers, but the ground truth label indicates that the subject has anxiety, the model's parameters are updated (e.g., via backpropagation) to improve its prediction accuracy. The training process may involve multiple iterations, with the diagnostic device 104 continuously refining the model until the model's predictions achieve a threshold level of accuracy.

Techniques operating according to the principles described herein may be implemented in any suitable manner. Included in the discussion above are a series of flow charts showing the steps and acts of various processes that generate a diagnostic output based on ambient audio data. The processing and decision blocks of the flow charts above represent steps and acts that may be included in algorithms that carry out these various processes. Algorithms derived from these processes may be implemented as software integrated with and directing the operation of one or more single- or multi-purpose processors, may be implemented as functionally-equivalent circuits such as a Digital Signal Processing (DSP) circuit, Field Programmable Gate Array (FPGA), or an Application-Specific Integrated Circuit (ASIC), or may be implemented in any other suitable manner. It should be appreciated that the flow charts included herein do not depict the syntax or operation of any particular circuit or of any particular programming language or type of programming language. Rather, the flow charts illustrate the functional information one of ordinary skill in the art may use to fabricate circuits or to implement computer software algorithms to perform the processing of a particular apparatus carrying out the types of techniques described herein. It should also be appreciated that, unless otherwise indicated herein, the particular sequence of steps and/or acts described in each flow chart is merely illustrative of the algorithms that may be implemented and can be varied in implementations and embodiments of the principles described herein.

Accordingly, in some embodiments, the techniques described herein may be embodied in computer-executable instructions implemented as software, including as application software, system software, firmware, middleware, embedded code, or any other suitable type of software. Such computer-executable instructions may be written using any of a number of suitable programming languages and/or programming or scripting tools, and also may be compiled as executable machine language code or intermediate code that is executed on a framework or virtual machine.

When techniques described herein are embodied as computer-executable instructions, these computer-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc. that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (i.e., as a single unit or separate units), or some of these functional facilities may not be implemented.

Computer-executable instructions implementing the techniques described herein (when implemented as one or more functional facilities or in any other manner) may, in some embodiments, be encoded on one or more computer-readable media to provide functionality to the media. Computer-readable media include magnetic media such as a hard disk drive, optical media such as a Compact Disk (CD) or a Digital Versatile Disk (DVD), a persistent or non-persistent solid-state memory (e.g., Flash memory, Magnetic RAM, etc.), or any other suitable storage media. Such a computer-readable medium may be implemented in any suitable manner, including as computer-readable storage media 606 of FIG. 6 described below (i.e., as a portion of a computing device 600) or as a stand-alone, separate storage medium. As used herein, “computer-readable media” (also called “computer-readable storage media”) refers to tangible storage media. Tangible storage media are non-transitory and have at least one physical, structural component. In a “computer-readable medium,” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium may be altered during a recording process.

In some, but not all, implementations in which the techniques may be embodied as computer-executable instructions, these instructions may be executed on one or more suitable computing device(s) operating in any suitable computer system, including the exemplary computer system of FIG. 1, or one or more computing devices (or one or more processors of one or more computing devices) may be programmed to execute the computer-executable instructions. A computing device or processor may be programmed to execute instructions when the instructions are stored in a manner accessible to the computing device/processor, such as in a local memory (e.g., an on-chip cache or instruction register, a computer-readable storage medium accessible via a bus, a computer-readable storage medium accessible via one or more networks and accessible by the device/processor, etc.). Functional facilities that comprise these computer-executable instructions may be integrated with and direct the operation of a single multi-purpose programmable digital computer apparatus, a coordinated system of two or more multi-purpose computer apparatuses sharing processing power and jointly carrying out the techniques described herein, a single computer apparatus or coordinated system of computer apparatuses (co-located or geographically distributed) dedicated to executing the techniques described herein, one or more Field-Programmable Gate Arrays (FPGAs) for carrying out the techniques described herein, or any other suitable system.

FIG. 6 illustrates one exemplary implementation of a computing device in the form of a computing device 600 that may be used in a system implementing the techniques described herein, although others are possible. It should be appreciated that FIG. 6 is intended neither to be a depiction of necessary components for a computing device to operate in accordance with the principles described herein, nor a comprehensive depiction.

Computing device 600 may comprise at least one processor 602, a network adapter 604, and computer-readable storage media 606. Computing device 600 may be, for example, a desktop or laptop personal computer, a personal digital assistant (PDA), a smart mobile phone, a server, a wireless access point or other networking element, or any other suitable computing device. Network adapter 604 may be any suitable hardware and/or software to enable the computing device 600 to communicate wired and/or wirelessly with any other suitable computing device over any suitable computing network. The computing network may include wireless access points, switches, routers, gateways, and/or other networking equipment as well as any suitable wired and/or wireless communication medium or media for exchanging data between two or more computers, including the Internet. Computer-readable storage media 606 may be adapted to store data to be processed and/or instructions to be executed by one or more processors 602. Processor 602 enables processing of data and execution of instructions. The data and instructions may be stored on the computer-readable storage media 606.

The data and instructions stored on computer-readable storage media 606 may comprise computer-executable instructions implementing techniques which operate according to the principles described herein. In the example of FIG. 6, computer-readable storage media 606 stores computer-executable instructions implementing various facilities and storing various information as described above. Computer-readable storage media 606 may store the various processes/facilities discussed above. In some embodiments, the diagnostic device 104 is a computing device 600 and the computer-readable storage media 606 may store the audio processing facility 116, audio buffer facility 118, and diagnostic facility 120. In some embodiments, the client computing device 102 is a computing device 600 and the computer-readable storage media 606 may store the audio capture facility 126 and the user interface facility 128. In some embodiments and the client computing device 102 are a computing device 600 and the computer-readable storage media 606 may store the audio processing facility 116, audio buffer facility 118, diagnostic facility 120, audio capture facility 126, and user interface facility 128.

While not illustrated in FIG. 6, a computing device may additionally have one or more components and peripherals, including input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computing device may receive input information through speech recognition or in other audible format.

Embodiments have been described where the techniques are implemented in circuitry and/or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

The word “exemplary” is used herein to mean serving as an example, instance, or illustration. Any embodiment, implementation, process, feature, etc., described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

What is claimed is:

1. A method comprising:
determining a prediction of whether a subject has any of one or more health conditions, wherein determining the prediction comprises:
in response to determining that ambient audio data includes a plurality of samples of speech of the subject having an aggregate duration greater than or equal to a threshold duration, determining the prediction based at least in part on an analysis of aggregated audio data of speech of the subject, the aggregated audio data comprising audio of the plurality of samples of speech of the subject,
wherein the analysis of the aggregated audio data comprises analyzing the ambient audio data using one or more trained models, the one or more trained models having been trained with training data including audio data of a plurality of prior subjects and information indicating whether each prior subject of the plurality of prior subjects had one or more health conditions; and
outputting the prediction of whether the subject has any of the one or more health conditions.

2. The method of claim 1, wherein determining the prediction based at least in part on an analysis of aggregated audio data of speech of the subject comprises:
extracting vocal biomarkers for the subject from the aggregated audio data of the speech of the subject; and
determining the prediction based at least in part on an analysis of the vocal biomarkers.

3. The method of claim 2, wherein analyzing the ambient audio data using one or more trained models comprises analyzing the vocal biomarkers of the subject using the one or more trained models.

4. The method of claim 3, wherein determining the prediction of whether the subject has any of the one or more health conditions comprises, for a health condition, generating a prediction of a severity of the health condition for the subject.

5. The method of claim 3, wherein determining the prediction of whether the subject has any of the one or more health conditions comprises, for a health condition, generating a prediction of a type of the health condition for the subject.

6. The method of claim 3, wherein determining the prediction of whether the subject has any of the one or more health conditions comprises generating a likelihood that the subject has a health condition.

7. The method of claim 1, further comprising:
receiving over time one or more segments of the ambient audio data, at least one of the segments of the ambient audio data including audio data of speech; and
identifying, from among the one or more segments of ambient audio data, the plurality of samples of speech of the subject.

8. The method of claim 7, wherein identifying the plurality of samples of speech of the subject from among the one or more segments of ambient audio data comprises:
determining, when a segment of ambient audio data comprises speech, whether the speech is speech of the subject;
in response to determining that a segment of ambient audio data comprises speech of the subject, determining whether at least some audio data of the speech of the subject included within the segment of ambient audio data satisfies one or more criteria for use as a speech sample; and
in response to determining that the at least some audio data of the speech of the subject satisfies the one or more criteria for use as a speech sample, including the at least some audio data as a sample of speech of the subject in the plurality of samples of speech of the subject.

9. The method of claim 8, wherein determining whether the speech is of the subject comprises:
when the ambient audio data comprises audio of speech of multiple speakers, identifying based on words spoken by the multiple speakers in the ambient audio data a role of each of the multiple speakers; and
identifying one of the speakers as the subject based on the role of each of the multiple speakers.

10. The method of claim 9, wherein determining, when a segment of ambient audio data comprises speech, whether the speech is speech of the subject comprises determining whether acoustic characteristics of the speech match acoustic characteristics for the speaker identified as the subject.

11. The method of claim 9, further comprising:
repeating the determining a prediction of whether a subject has any of one or more health conditions for each of at least one other speaker of the multiple speakers.

12. The method of claim 8, wherein determining whether the speech is of the subject comprises:
when the ambient audio data comprises audio of speech of multiple speakers, identifying whether any of the multiple speakers matches one or more known acoustic characteristics of speech.

13. The method of claim 12, wherein determining whether the speech is of the subject further comprises:
identifying, as the subject, a speaker for which speech does not match known acoustic characteristics of speech.

14. The method of claim 8, wherein:
the threshold duration is a first threshold duration; and
determining whether the at least some audio data of the speech satisfies one or more criteria for use as a speech sample comprises determining whether the at least some audio data of the speech has a duration longer than a second threshold duration.

15. The method of claim 8, wherein determining whether the at least some audio data of the speech satisfies one or more criteria for use as a speech sample comprises determining whether the at least some audio data of the speech comprises audio of the subject speaking a plurality of different words.

16. The method of claim 8, wherein determining whether the at least some audio data of the speech satisfies one or more criteria for use as a speech sample comprises evaluating a meaning of words included in the speech of the subject.

17. The method of claim 8, wherein determining whether the at least some audio data of the speech satisfies one or more criteria for use as a speech sample comprises evaluating an acoustic quality of the at least some audio data.

18. The method of claim 7, wherein determining the prediction of whether the subject has any of one or more health conditions comprises:
iteratively repeating over the time the determining the prediction of whether the subject has the one or more health conditions, wherein in each iteration of the iteratively repeating, the determining the prediction is performed using a different portion of the one or more segments of ambient audio data received over the time.

19. The method of claim 18, wherein:
the iteratively repeating comprises at least a first iteration and a second iteration;
in the first iteration, the determining the prediction is performed using a first set of segments of ambient audio data, the first set of segments of ambient audio data being fewer than all of the segments of ambient audio data received over the time;
in the second iteration, the determining the prediction is performed using a second set of segments of ambient audio data, the second set of segments of ambient audio data being fewer than all of the segments of ambient audio data received over the time; and
the first and second sets of segments of audio data partially overlap.

20. The method of claim 19, wherein the first iteration and second iteration are performed contemporaneous with speaking by the subject of speech of the first and second sets of segments of ambient audio data.

21. The method of claim 7, wherein the determining is performed contemporaneous with a speaking by the subject of the speech.

22. The method of claim 7, wherein identifying the plurality of samples of speech of the subject from among the one or more segments of ambient audio data comprises:
identifying multiple speakers for which at least some of the segments of ambient audio data contains speech; and
identifying the subject as one of the multiple speakers.

23. The method of claim 7, wherein the one or more segments of the ambient audio data is a plurality of segments of audio data, and at least some of the segments of the ambient audio data do not include audio of speech.

24. The method of claim 1, wherein:
the ambient audio data comprises two or more audio channels; and
the method further comprises identifying a channel, from among the two or more audio channels, containing speech of the subject.

25. The method of claim 24, wherein identifying the channel comprising speech of the subject, from among the two or more audio channels, comprises identifying the channel based at least in part on speaker role identifiers corresponding to each audio channel of the two or more channels.

26. The method of claim 1, further comprising:
receiving a segment of the ambient audio data; and
obtaining one or more of the plurality of samples of speech from the segment of ambient audio data.

27. The method of claim 26, wherein obtaining one or more of the plurality of samples of speech from the segment comprises receiving all of the plurality of samples of speech from the segment of ambient audio data.

28. The method of claim 1, further comprising:
outputting, together with the prediction, an indication of an activity in which the subject was engaged when the plurality of samples of speech were captured.

29. The method of claim 1, further comprising:
receiving the ambient audio data, the ambient audio data having been captured during a clinical encounter between the subject and a clinician.

30. The method of claim 29, wherein outputting the prediction comprises outputting the prediction for presentation to the clinician during the clinical encounter.

31. The method of claim 1, further comprising:
receiving the ambient audio data, the ambient audio data having been captured during at least one time the subject was speaking to another person within range of a microphone.

32. The method of claim 31, wherein outputting the prediction comprises outputting the prediction to the subject and/or to the other person.

33. The method of claim 32, wherein outputting the prediction comprises outputting the prediction to the other person during a time the subject was speaking to the other person.

34. At least one computer-readable storage medium storing computer-executable instructions that, when executed by at least one processor, cause the at least one processor to carry out a method comprising:
determining a prediction of whether a subject has any of one or more health conditions, wherein determining the prediction comprises:
in response to determining that ambient audio data includes a plurality of samples of speech of the subject having an aggregate duration greater than or equal to a threshold duration, determining the prediction based at least in part on an analysis of aggregated audio of speech of the subject, the aggregated audio data comprising audio of the plurality of samples of speech of the subject,
wherein the analysis of the aggregated audio data comprises analyzing the ambient audio data using one or more trained models, the one or more trained models having been trained with training data including audio data of a plurality of prior subjects and information indicating whether each prior subject of the plurality of prior subjects had one or more health conditions; and
outputting the prediction of whether the subject has any of the one or more health conditions.

35. An apparatus comprising:
at least one processor; and
at least one storage medium having stored thereon executable instructions that, when executed by the at least one processor, cause the at least one processor to carry out a method of predicting a health condition in a subject, the method comprising:
determining a prediction of whether the subject has any of one or more health conditions, wherein determining the prediction comprises:
in response to determining that ambient audio data includes a plurality of samples of speech of the subject having an aggregate duration greater than or equal to a threshold duration, determining the prediction based at least in part on an analysis of aggregated audio of speech of the subject, the aggregated audio data comprising audio of the plurality of samples of speech of the subject,
wherein the analysis of the aggregated audio data comprises analyzing the ambient audio data using one or more trained models, the one or more trained models having been trained with training data including audio data of a plurality of prior subjects and information indicating whether each prior subject of the plurality of prior subjects had one or more health conditions; and
outputting the prediction of whether the subject has any of the one or more health conditions.
